(19) United States
(12) Patent Application Publication
(10) Pub. No.: US 2014/0207074 A1
(43) Pub. Date: Jul. 24, 2014

(54) DRUG DELIVERY DEVICE WITH MEANS FOR HANDLING DATA

(71) Applicant: NOVO NORDISK A/S, Bagsvaerd (DK)

(72) Inventor: Ole Christian Nielsen, Hilleroed (DK)

(21) Appl. No.: 14/239,025

(22) PCT Filed: Aug. 17, 2012
(86) PCT No.: PCT/EP2012/066122

§ 371 Date: Apr. 4, 2014

Related U.S. Application Data
(60) Provisional application No. 61/526,841, filed on Aug. 24, 2011.

(30) Foreign Application Priority Data
Aug. 18, 2011 (EP) 11177926.0

(51) Int. Cl. A61M 5/315

(52) U.S. Cl. CPC A61M 5/31533
USPC 604/189; 604/207

(57) ABSTRACT

A system comprises a drug reservoir and means for expelling drug from the reservoir, the drug expelling means comprising setting means allowing a user to set a drug dose to be expelled from the reservoir, and actuation means for driving or releasing the drug expelling means to expel the set dose amount. The system further comprises a first electronic data processing system adapted to capture and store data representing a property related to the amount of drug expelled from the reservoir by the expelling means, and a second electronic data processing system adapted to receive and store data from an external source, wherein the system is configured to not allow code or data processed by the second electronic data processing system to be processed by the first electronic data processing system.

The present invention relates to equipment for handling data in a drug delivery. Especially, the invention addresses the issue of safely handling data generated by a drug delivery device as well as data supplied to the drug delivery device from an external source.

### BACKGROUND OF THE INVENTION

In the disclosure of the present invention reference is mostly made to the treatment of diabetes by delivery of insulin, however, this is only an exemplary use of the present invention.

Drug Injection devices have greatly improved the lives of patients who must self-administer drugs and biological agents. Drug Injection devices may take many forms, including simple disposable devices that are little more than an ampoule with an injection means or they may be durable devices adapted to be used with prefilled cartridges. Regardless of their form and type, they have proven to be great aids in assisting patients to self-administer injectable drugs and biological agents. They also greatly assist care givers in administering injectable medicines to those incapable of performing self-injections.

Performing the necessary insulin injection at the right time and in the right size is essential for managing diabetes, i.e. compliance with the specified insulin regimen is important. In order to make it possible for medical personnel to determine the effectiveness of a prescribed dosage pattern, diabetes patients are encouraged to keep a log of the size and time of each injection. However, such logs are normally kept in handwritten notebooks, from the logged information may not be easily uploaded to a computer for data processing. Therefore, it is desirable to automate the logging of ejection information from medication delivery systems. Correspondingly, injection devices for self-injections have been proposed which integrate this monitoring/acquisition mechanism into the device itself, either for a per se electronically controlled device such as a motor doser, or for a per se mechanically operated drug delivery device which may be either of the durable type as disclosed in e.g. U.S. Pat. No. 6,585,698, US 2009/0318865 and US 2011/0270214, which are hereby incorporated by reference, or the prefilled type. The latter devices are to be discarded after being emptied and so inexpensive that it is not cost-effective to build-in electronic data acquisition functionality in the device it-self. Correspondingly, data acquisition/monitoring functionality have been proposed to be provided in a separate device to be put on or in the injection device, i.e. some kind of accessory e.g. an add-on module to the injection device. For example, WO 2010/098927 discloses a medical module which is configured to be attached to a drug delivery pen, the module being adapted to detect and store selected and ejected dosages as well as other data.

For many patients on medical treatment with injection devices a yet better compliance to their prescribed treatment and therapy would be possible if they could be supported by a number of guidance, support and complementing systems, e.g. displayed information of where to inject, displayed information of how to prime, as well as dosing suggestions based on dietary, blood glucose measurements and input provided by the patient or another apparatus. In the article “The Smart Insulin Pen” published on diabetesnet.com a drug delivery device is proposed which may log dose data as well as contain a food database with data transmitted from a PC.

Having regard to the above, it is an object of the present invention to provide devices and methods which safely supports and guides a user of a drug delivery device provided with electronics for capturing and processing data and code

### DISCLOSURE OF THE INVENTION

In the disclosure of the present invention, embodiments and aspects will be described which will address one or more of the above objects or which will address objects apparent from the below disclosure as well as from the description of exemplary embodiments.

For a number of the functions wanted on an injection device an electronic communication port is required, however when implementing such a communication port there is always a risk to interfere with the devices primary safety related functions, e.g. capturing dose data representing amounts of drug expelled by the drug delivery device. This risk is much increased if the electronic communication port is also wanted for update of the program itself or for additions or alterations to the program controlling the micro controller system implemented in the device. But a number of functions are often wanted on a “download when needed” requirement just like applets in a mobile phone network to deal with specific tasks for a longer or shorter period. The problem with such a system able to add or change the program of its micro controller is that it could jeopardize the primary functions of the injection device if containing malware or errors.

Thus, in a first aspect of the invention a drug delivery device is provided, comprising a drug reservoir or means for receiving a drug reservoir, and drug expelling means for expelling an amount of drug from the reservoir, drug expelling means comprising setting means allowing a user to set a dose amount to be expelled from the drug reservoir, and actuation means for driving or releasing the drug expelling means to expel the set dose amount. The device further comprises a first electronic data processing system adapted to capture and store data representing a property related to the amount of drug expelled from the reservoir by the expelling means, and a second electronic data processing system adapted to receive and store data from an external source, wherein the device is configured to not allow code or data processed by the second electronic data processing system to be processed by the first electronic data processing system.

The device may be configured to not allow code or data to be transferred from the second to the first electronic data processing system, e.g. by hardware design, or code and data transfer between the first and second electronic data processing system may be controlled by validated software embedded in the first electronic data processing system, the validated software preventing that code and data from the second electronic data processing system is processed by the first electronic data processing system. The first electronic data processing system may comprise a first processor, and the second electronic data system may comprise a second processor. The second electronic data processing system may be adapted to receive program instructions allowing, e.g. user-chosen application software to be run on the system or an update of a program or part of a program to another version.

In an exemplary embodiment the first electronic data processing system comprises a first display controlled by the first electronic data processing system to display dose values for drug expelled from the reservoir by the expelling means, and the second electronic data processing system comprises a second display controlled by the second electronic data processing system, either as two separate displays or incorporated in a single display unit. The second electronic data processing system may be adapted to receive program instructions and user input data allowing e.g. a recommended drug dose to be calculated and displayed on the second display or it may be used to participate in social networks.

The drug delivery device may further comprise first transmission means controlled by the first electronic data processing system for transmitting data captured by the first electronic data processing system, and/or second transmission means controlled by the second electronic data processing system for transmitting data processed by the second electronic data processing system. The transmission means may be adapted for wired or wireless transmission of data.

The drug delivery device may be of a modular design comprising e.g. a drug delivery unit and a combined data capture and data management unit releasably attachable to each other, this allowing the drug delivery unit to be of a disposable pre-filled design, or the drug delivery device may be in the form a unitary drug delivery device comprising integrated data capture means.

As used herein, the term “insulin” is meant to encompass any drug-containing flowable medicine capable of being passed through a delivery means such as a cannula or hollow needle in a controlled manner, such as a liquid, solution, gel or fine suspension, and which has a blood glucose controlling effect, e.g. human insulin and analogues thereof as well as non-insulins such as GLP-1 and analogues thereof. In the description of the exemplary embodiments reference will be made to the use of insulin.

### BRIEF DESCRIPTION OF THE DRAWINGS

In the following the invention will be further described with reference to the drawings, wherein

FIG. 1 shows a pen-type drug delivery device comprising electronic means adapted for data processing,

FIG. 2 shows a schematic representation of an architecture for a drug delivery device, and

FIG. 3 shows a schematic representation of a drug delivery device in combination with a mobile phone.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only.

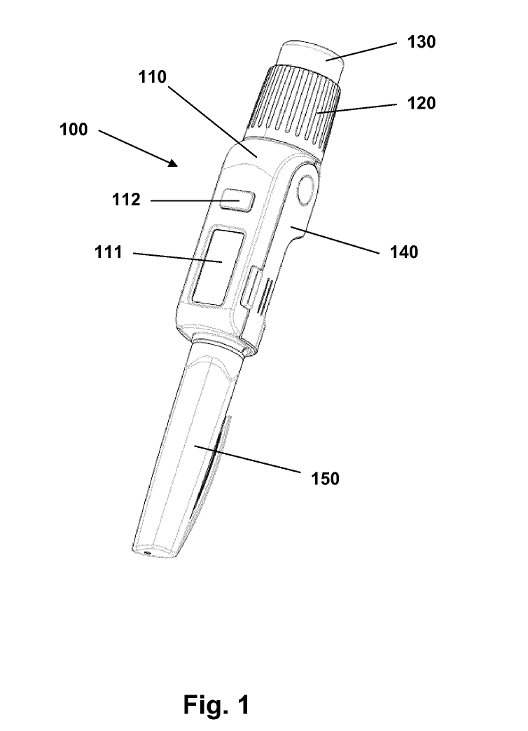

FIG. 1 shows an embodiment of a modular drug delivery device comprising a pen-formed drug delivery unit and a combined data capture and data management unit releasably attachable to each other, however, the figure could also represent a unitary drug delivery device comprising integrated data capture means. The drug delivery unit per se could be of any desirable design providing the necessary input to the combined data capture and data management unit, however, the shown embodiment represents the type of pen in which a spring is loaded during setting of a dose, this allowing spring-driven drug expelling when the set and loaded mechanism is released by the user, this allowing a design in which the release (or actuation) button is axially stationary during dose setting. A more detailed description of such a pen can be found in e.g. US 2009/054839, US 2008/306446 and US 2008/234634 which are hereby incorporated by reference.

More specifically, such a drug delivery device of the pen type comprises a proximal part having a mainly cylindrical housing portion with an expelling mechanism and a distal portion comprising a drug cartridge with an axially moveable piston driven by the expelling mechanism. The pen comprises a rotatable dose setting ring member allowing a user to set and adjust (i.e. dial-up and dial-down) a variable dose size of given increments (e.g. 1 IU insulin) to be expelled from the cartridge, the actual dose size (e.g. 25 IU insulin) being indicated by numbers shown in a window, the numbers being arranged on a rotating dose drum member (not to be seen in FIG. 1). The maximum amount of drug that can be delivered during one outdosing is defined by the injection device. For example the injection device may deliver variable dose amounts during one out-dosing between 1 IU insulin and 80 IU insulin. A push button is arranged at the proximal end and adapted to release the expelling mechanism when pushed distally by the user. As the mechanism is released the set dose will be expelled from the cartridge and the dose drum will correspondingly rotate back to its initial zero position. If the mechanism is designed to stop expelling when the user stops pushing the release button, the number display in the window will show the portion of the dose (e.g. the numbers of units) not yet expelled, e.g. 10 units of insulin.

Based on a drug delivery pen of the type described above FIG. 1 shows an embodiment of a modular drug delivery device 100 comprising a pen-formed drug delivery unit and a combined data capture and data management unit, the units being firmly but releasably attachable to each other. The device comprises a rotatable dose setting member 120 and a proximally arranged release button 130 adapted to be moved between an initial position, an intermediate position, and an actuated position in which the expelling means is actuated to expel the set dose. The distal reservoir part of the drug delivery device is covered by a cap member 150. The combined data capture and data management unit 110 comprises electronic detection means for capturing data representing a property related to the amount of drug expelled from the reservoir by the expelling means (see below), and switch means for initiating data capture, the switch means being actuated when the release button is moved from its initial to its intermediate position. The combined data capture and data management unit also comprises a combined display 111 adapted to show e.g. time and dose size for the last expelling action, as well as a button 112 allowing a user to e.g. toggle between a number of recent time-dose logs and/or other functions. The combined data capture and data management unit is further provided with an input port for wired or wireless download of data from an external device, just as it may be provided with an output port for uploading data e.g. to the users smartphone or a doctors PC.

For example, in addition to the data capture functionality the drug delivery device of FIG. 1 may be provided with a bolus calculator which has been downloaded to the device, this providing a bolus calculator which is up-to-date in respect of both the algorithms implemented as well as the features offered. The device may further be adapted to receive blood glucose (BG) data from a BG meter or, alternatively, comprise a BG meter. As the system is provided with a very simple user interface having only one button, meal input data to the system may be downloaded from e.g. a smartphone, such a device providing ease of entering meal data as well as other meal related data.

In case a drug delivery device is provided in the form of a motor doser typically having a box-shaped configuration, it would be possible to provide the device with a relatively large touch display allowing for easy direct input of user generated data, e.g. meal related data. Further, with a larger display it would also be relevant to provide more advanced features such as a diary presenting data, e.g. BG values and dose data, in graphical form. Such a diary feature could also be provided as downloadable “app” software.

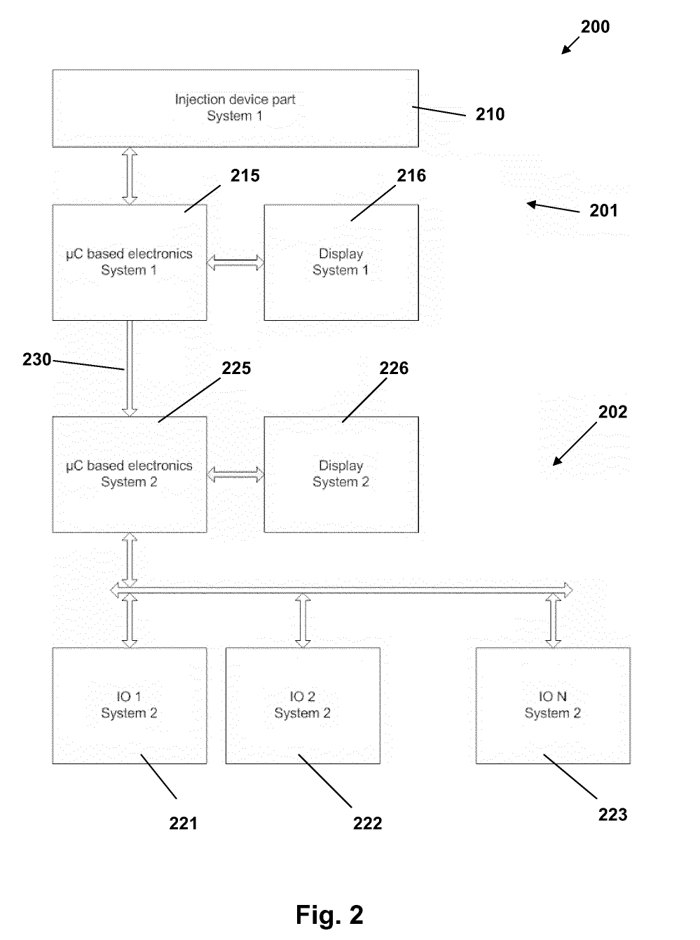

FIG. 2 shows a schematic representation of an exemplary architecture for a drug delivery device comprising an electronic system providing a high degree of safety.

More specifically, the system 200 comprises two subsystems, a first system 201 comprising an injection device part 210 and associated electronics and a second system 202 mainly comprising electronics. The injection device part comprises a drug reservoir (or means for receiving a drug reservoir) and drug expelling means for expelling an amount of drug from the reservoir, the drug expelling means comprising setting means allowing a user to set a dose amount to be expelled from the drug reservoir, and actuation means for driving or releasing the drug expelling means to expel the set dose amount. The injection device part may be of the mechanical type as described with respect to FIG. 1 or it may be of the electronically controlled motor doser type. The first system further comprises a first micro processer controlled electronic data processing system 215 adapted to capture and store data representing a property related to the amount of drug expelled from the reservoir by the expelling means, e.g. number of units of insulin, as well as a first display 216 controlled by the first micro processer controlled electronic data processing system to indicate to a user e.g.

the number of units of insulin expelled. The first system also comprises an interface between the delivery and electronics system which for a mechanical expelling assembly could be e.g. positional switches, optical transducers and/or other digital and/or analogue electronics parts able to convert mechanical information to electronics information and/or vice verse in order to provide input and/or output to control the injection device. The first system may further be provided with first transmission means (not shown) controlled by the first electronic data processing system for wired or wirelessly transmitting data captured by the first electronic data processing system.

The second system 202 comprises a second micro processer controlled electronic data processing system 225 adapted to receive and store data from one or more external sources, as well as a second display 226 controlled by the second micro processer controlled electronic data processing system to display information to a user. For example, the second electronic data processing system may be adapted to receive program instructions, BG data and user input data via a number of I/O input-output ports 221, 222, 223 allowing e.g. a recommended drug dose to be calculated and displayed on the second display. The second system may further be provided with second transmission means (not shown) controlled by the second electronic data processing system for wired or wirelessly transmitting data processed by the second electronic data processing system.

As indicated by the one-way arrow 230 the combined system is configured to not allow code or data processed by the second electronic data processing system to be processed by the first electronic data processing system. For example, the system may be configured to not allow code or data to be transferred from the second to the first electronic data processing system, e.g. by a design in which data transfer between the first and second electronic systems is controlled by hardware design in the form of a one-way data transfer port, e.g. a uni-directional FIFO (first in first out buffer) able to transfer data from system 1 to system 2 but not able to transfer code or data the opposite way or implemented with a parallel dual ported RAM (Random Access Memory) with supporting allocation hardware for making the system 1 only able to write and system 2 only able to read from this communication means. Code and data transfer between the first and second electronic data processing system may also be controlled by validated software embedded in the first electronic data processing system, the validated software preventing that code and data from the second electronic data processing system is processed by the first electronic data processing system. The validated code in system 1 can be implemented on a microprocessor with a standard communication port as e.g. a SPI port (Serial Peripheral Interface Bus). The validation assures that the safety requirements for a given application or market can be met in an objective way by the system conforming to a given validation standard, e.g. ISO/EN 14971 used for risk management for medical devices.

The first and second electronic data processing systems may be designed to share some of the electronic circuitry, e.g. they may be provided with a common energy source. Given that the requirements to the prevention of data and code transfer between the two subsystems are met, the system may share further components such as part of memory and processor resources. To indicate to a user that different kinds of data may represent different levels of data safety, the above-described two displays may be arranged to directly show this. For example, the two displays may be fully separate or they may be provided by dividing a common display member into two dedicated areas, one for each subsystem.

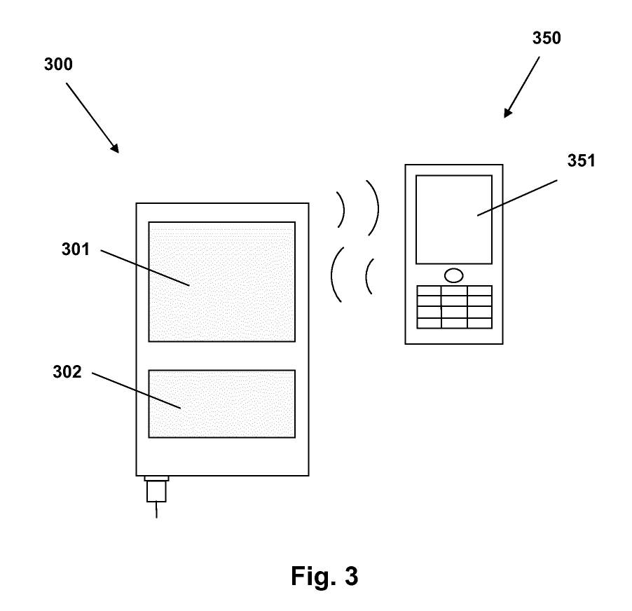

FIG. 3 shows an electronically controlled motor doser 300 in combination with an external electronic device 350 in the form of a mobile phone. In the shown embodiment the motor doser is provided with relatively large displays 301, 302 and the mobile phone with a relatively small display 351, however, this is just an example. As indicated, the two units are adapted for two-ways wireless communication.

Communication could be used to transfer data for a number of purposes. For example, the mobile phone could be used as a means to download application software, e.g. from the Internet, which is then transmitted to the second “open” part of the electronic control system of the motor doser. Correspondingly, information captured by the first “closed” part of the electronic control system of the motor doser could be transferred to the mobile phone for subsequent upload to e.g. the patients doctor, for example in combination with data from the open part, e.g. BG values transferred to the motor doser from a BG meter (not shown).

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A drug delivery device comprising:
a drug reservoir or means for receiving a drug reservoir,
drug expelling structure to expel an amount of drug from the reservoir, comprising:
setting structure allowing a user to set a dose amount to be expelled from the drug reservoir, and
actuation structure for driving or releasing the drug expelling structure to expel the set dose amount,
a first electronic data processing system adapted to capture and store data representing a property related to the amount of drug expelled from the reservoir by the expelling structure, and
a second electronic data processing system adapted to receive and store data from an external source,
wherein the drug delivery device is configured to not allow code or data to be transferred from the second to the first electronic data processing system, and
wherein data transfer between the first and second electronic systems is controlled by hardware design.

2. A drug delivery device as in claim 1, wherein the first electronic data processing system comprises a first processor, and the second electronic data system comprises a second processor.

3. A drug delivery device as in claim 1, wherein the second electronic data processing system is adapted to receive program instructions.

4. A drug delivery device as in claim 1, wherein:
the first electronic data processing system comprises a first display controlled by the first electronic data processing system to display dose values for drug expelled from the reservoir by the expelling structure, and
the second electronic data processing system comprises a second display controlled by the second electronic data processing system.

5. A drug delivery device as in claim 4, wherein the first and second displays are incorporated in a single display unit.

6. A drug delivery device as in claim 4, wherein the second electronic data processing system is adapted to receive program instructions and user input data allowing a recommended drug dose to be calculated and displayed on the second display.

7. A drug delivery device as in claim 5, further comprising:
first transmission structure controlled by the first electronic data processing system for transmitting data captured by the first electronic data processing system.

8. A drug delivery device as in claim 7, wherein the first transmission structure is adapted for wireless transmission of data.

9. A drug delivery device as in claim 7, further comprising:
second transmission structure controlled by the second electronic data processing system for transmitting data processed by the second electronic data processing system.

10. A drug delivery device as in claim 9, wherein the second transmission structure is adapted for wireless transmission of data.
